SEQUENCE LISTING (1 sequence)

SEQ ID NO: 1 - DNA, 29 bp, Homo sapiens
ttcaaatatgtatccgctcatgagacaat

(19) United States
(12) Patent Application Publication
(10) Pub. No.: US 2016/0130588 A1
(43) Pub. Date: May 12, 2016

(54) METHOD FOR THE GENERATION OF POLYCYSTRONIC VECTORS

(71) Applicants: Wassim Abdul RAHMAN, Basel (CH); Nicolas Holger THOMÄ, Basel (CH)

(72) Inventors: Wassim Abdul RAHMAN, Basel (CH); Nicolas Holger THOMÄ, Basel (CH)

(73) Assignee: FRIEDRICH MIESCHER INSTITUTE FOR BIOMEDICAL RESEARCH (FMI), Basel (CH)

(21) Appl. No.: 14/776,076

(22) PCT Filed: Mar. 14, 2014
(86) PCT No.: PCT/IB2014/059792

§ 371 Date: Sep. 14, 2015

(30) Foreign Application Priority Data
Mar. 14, 2013 (EP) 13159307.1

(51) Int. Cl. C12N 15/64

(52) U.S. Cl. CPC C12N 15/64

(57) ABSTRACT

The present invention provides a method for generating polycystronic nucleic acid vectors, said method comprising the steps of a) providing a first nucleic acid vector, said first nucleic acid vector comprising: i) an origin of replication placed in front of ii) at least one first gene of interest functionally cloned within a first expression cassette, said first expression cassette comprising a promoter sequence as well as a termination sequence, said first gene of interest being located between said promoter sequence and said termination sequence, iii) a marker gene together with its termination sequence, said marker gene being situated downstream of a target sequence for a recombinase, wherein the promoter necessary for the expression of said marker gene in a host cell is absent from said first nucleic acid vector, and optionally iv) a first further marker gene which is different from the marker gene of iii), said first further marker gene being situated within said first expression cassette of ii) or within a further expression cassette, said first further marker gene allowing for the selection of cells comprising said first nucleic acid vector during the process of generating said first nucleic acid vector; b) providing a second nucleic acid vector, said second nucleic acid vector comprising i) an origin of replication placed in front of ii) at least one second gene of interest functionally cloned within a second expression cassette, said second expression cassette comprising a promoter sequence as well as a termination sequence, said second gene of interest being located between said promoter sequence and said termination sequence, iii) a promoter situated upstream of the same target sequence for a recombinase as present in front of the marker gene of iii) in the first nucleic acid vector, wherein said promoter is suitable for the expression in a host cell of said marker comprised in the first nucleic acid vector gene, and iv) optionally a second further marker gene which is different from the marker gene of iii), second further marker gene being situated within said second expression cassette of ii) or within a further expression cassette, said second further marker gene allowing for the selection of cells comprising said first nucleic acid vector during the process of generating said second nucleic acid vector; and c) contacting, under conditions suitable for a recombination to take place, a mixture of the first and the second nucleic acid vectors with a recombinase, which recombinase specifically recombines the target sequences of iii) which is situated downstream of the promoter of the second nucleic acid vector and upstream of the marker gene of the first nucleic acid vector, respectively.

### BACKGROUND OF THE INVENTION

For expression purposes recombinant genes are usually transfected into the target cells, cell populations or tissues, as cDNA constructs in the context of an active expression cassette to allow transcription of the heterologous gene. The DNA construct is recognized by the cellular transcription machinery in a process that involves the activity of many trans-acting transcription factors (TF) at cis-regulatory elements, including enhancers, silencers, insulators and promoters (herein globally referred to as “promoters”). The expression of single recombinant genes is well known in the art. There is however a need for tools to study multiprotein complexes.

The identification of novel multiprotein complexes in eukaryotic cells has accelerated considerably, in particular due to the advent of genome-wide analysis of protein-protein interactions, the introduction of powerful multiple-affinity protein purification methods and the development of ultrasensitive analytical techniques. Based on extensive two-hybrid searches, the number of interaction partners for a given protein was estimated for baker's yeast to range on average from five to eight (e.g. Schwikowski et al., 2000, Nat. Biotechnol. 18:1257-1261; Bader et al. 2002, Nat. Biotechnol. 20:991-997 (2002); Von Mering et al. 2002, Nature 417:399-403; Grigoriev, A. 2003, Nucleic Acids Res. 31:4157-4161). The concept of the cell as a collection of multicomponent protein machines, one for essentially every major process, has thereby emerged. As explained in e.g. WO-A-2005/085456 or in WO-A-2007/054250, this poses significant challenges for protein production technologies aimed at molecular level structural and functional studies of eukaryotic multiprotein complexes as intracellular quantities are typically refractory to large-scale extraction from source.

Polycistronic vectors carrying several genes encoding multiprotein complex components have been used for selected cases of delivery in gene therapy (De Felipe, P. 2002, Curr. Gene Ther. 2:355-378; Planelles, V. 2003, Methods Mol. Biol. 229:273-284). An example of a polycistronic vector used for the expression of a transcription factor complex composed of four subunits in E. coli can be found in Selleck et al. 2001, Nat. Struct. Biol. 8:695-700. The DNA constructs in these studies were generated by conventional methods using endonucleases and ligase. Protein complexes in eukaryotes, however, often contain ten or more subunits with individual polypeptides ranging in size up to several hundred kDa, which severely restricts the applicability of conventional cloning strategies and usually rules out E. coli as a useful host system for heterologous protein expression.

Recombinant baculoviruses are particularly attractive for high-level production of large protein assemblies (O'Reilly et al. Baculovirus expression vectors. A laboratory manual. Oxford Press, New York (1994)). Genes driven by Autographs californica nuclear polyhedrosis virus (AcNVP) late promoters are often abundantly expressed, authentically processed, and targeted to their appropriate cellular compartment. Even architecturally complex particles such as capsid structures have been successfully assembled in insect cells using the baculovirus system (Roy et al., 1997, Gene 190:119-129). Expression of several genes in one cell can be achieved by co-infection with viruses carrying one foreign gene each. However, this approach inevitably leads to considerable variations in individual protein production levels as a result of statistical variance in virus stochiometry between infected cells. A superior alternative is infection with one baculovirus containing all heterologous genes of choice (Roy et al., 1997, Gene 190:119-129; Bertolotti-Ciarlet et al. 2003, Vaccine 21:3885-3900). This is possible due to the flexible nature of the AcNVP envelope which allows for accommodation of large DNA insertions into the circular 130 kb dsDNA baculovirus genome. Traditionally, recombinant baculovirus generation is carried out in two steps. Foreign genes are first cloned into a small transfer vector propagated in E. coli and then inserted into the large baculovirus genome by homologous recombination in insect cells in a reaction yielding 30-80% recombinant progeny if linearized parent viral DNA is used (O'Reilly et al. Baculovirus expression vectors. A laboratory manual. Oxford Press, New York (1994)). This procedure was substantially simplified by the introduction of a baculovirus shuttle vector (bacmid bMON14272) propagated in E. coli (Luckow et al. 1993, J. Virol. 67:4566-4579). The bacmid contains the Tn7 attachment site for transposition of foreign genes from a transfer vector (Bac-to-Bac™, Invitrogen: Bac-to-Bac™ Baculovirus Expression Systems Manual, Invitrogen, Life technologies Incorporated (2000)). A bicistronic transfer vector, pFastBac™ Dual, containing polyhedrin (polh) and p10 late viral promoters in two separate expression cassettes with sets of restriction sites for sequential subcloning of two foreign genes for co-expression was introduced. For the production of multiprotein complexes containing large as well as many subunits, there still is a strong need for transfer vectors that allow for assembling genes in a non-sequential, modular manner to alleviate limitations imposed by the paucity of useful restriction sites in a collection of coding sequences each encompassing several thousand base pairs.

A solution to this need has been proposed in WO-A-2005/085456. The methods proposed in this document however still have disadvantages. The vectors of the system carry distinct replication origins requiring plasmid handling/amplification in different bacterial strains leading to significantly different plasmid production yields. Hence, bacteria cultures for plasmid production have to be performed at different scales depending on each plasmid. In addition, the vectors carry resistance genes for distinct antibiotics. These limitations require handling of the initial vectors in distinct conditions and make the system incompatible with e.g. in-parallel/robotized strategies. Therefore, it is an objective of the present invention to provide new tools and methods for the rapid and flexible generation of multiprotein complexes.

It is a further objective of the present invention to provide new tools and methods for generating large multiprotein complexes with many subunits and at the same time circumventing the requirements for unique restriction sites.

The present invention also provides for a highly flexible system for multiprotein complex production where the encoding sequences for individual subunits can be substituted in a modular manner avoiding de novo regeneration of the entire ensemble of encoding sequences.

There is also a need for tools and methods where easy, modular exchange of organism-, tissue- or cell type-specific promoters is possible, for recombinantly producing a particular multiprotein complex in various organisms, eukaryotic (mammalian, yeast, etc.) and prokaryotic, or under chosen conditions (e.g. early, late, very late phases of viral infection of a host cell, etc.).

The reason for this need is that the solubility and activity of biological macromolecules is often compromised if the interacting components are produced and purified in isolation, and reconstitution under in vitro conditions often does not produce physiologically meaningful data. Therefore, for the investigation of the interplay between macromolecular complexes among each other, there is a need for powerful and expandable systems for enabling co-expression of two or several protein assemblies ideally in one cell, each consisting of several components.

### SUMMARY OF THE INVENTION

The present inventors have now surprisingly found that it is possible to insert a target sequence for a recombinase between a gene and its promoter, thus allowing the generation of multicistronic nucleic acid vectors.

The present invention hence provides a method for generating polycystronic nucleic acid vectors, said method comprising the steps of a) providing a first nucleic acid vector, said first nucleic acid vector comprising: i) an origin of replication placed in front of ii) at least one first gene of interest functionally cloned within a first expression cassette, said first expression cassette comprising a promoter sequence as well as a termination sequence, said first gene of interest being located between said promoter sequence and said termination sequence, iii) a marker gene together with its termination sequence, said marker gene being situated downstream of a target sequence for a recombinase, wherein the promoter necessary for the expression of said marker gene in a host cell is absent from said first nucleic acid vector, and optionally iv) a first further marker gene which is different from the marker gene of iii), said first further marker gene being situated within said first expression cassette of ii) or within a further expression cassette, said first further marker gene allowing for the selection of cells comprising said first nucleic acid vector during the process of generating said first nucleic acid vector; b) providing a second nucleic acid vector, said second nucleic acid vector comprising i) an origin of replication placed in front of ii) at least one second gene of interest functionally cloned within a second expression cassette, said second expression cassette comprising a promoter sequence as well as a termination sequence, said second gene of interest being located between said promoter sequence and said termination sequence, iii) a promoter situated upstream of the same target sequence for a recombinase as present in front of the marker gene of iii) in the first nucleic acid vector, wherein said promoter is suitable for the expression in a host cell of said marker comprised in the first nucleic acid vector gene, and iv) optionally a second further marker gene which is different from the marker gene of iii), second further marker gene being situated within said second expression cassette of ii) or within a further expression cassette, said second further marker gene allowing for the selection of cells comprising said first nucleic acid vector during the process of generating said second nucleic acid vector; and c) contacting, under conditions suitable for a recombination to take place, a mixture of the first and the second nucleic acid vectors with a recombinase, which recombinase specifically recombines the target sequences of iii) which is situated downstream of the promoter of the second nucleic acid vector and upstream of the marker gene of the first nucleic acid vector, respectively.

The present invention also provides a kit of parts comprising a) a first nucleic acid vector, said first nucleic acid vector comprising i) an origin of replication placed in front of ii) at least one first gene of interest functionally cloned within a first expression cassette, said first expression cassette comprising a promoter sequence as well as a termination sequence, said first gene of interest being located between said promoter sequence and said termination sequence, iii) a marker gene together with its termination sequence, said marker gene being situated downstream of a target sequence for a recombinase, wherein the promoter necessary for the expression of said marker gene in a host cell is absent from said first nucleic acid vector, and iv) optionally a first further marker gene which is different from the marker gene of iii), said first further marker gene situated within said first further marker gene allowing for the selection of cells comprising said first nucleic acid vector during the process of generating said first nucleic acid vector; and b) a second nucleic acid vector, said second nucleic acid vector comprising i) an origin of replication placed in front of ii) at least one second gene of interest functionally cloned within a second expression cassette, said second expression cassette comprising a promoter sequence as well as a termination sequence, said second gene of interest being located between said promoter sequence and said termination sequence, iii) a promoter situated upstream of the same target sequence for a recombinase as present in front of the marker gene in the first nucleic acid vector, wherein said promoter is suitable for the expression in a host cell of the marker comprised in the first nucleic acid vector gene, and iv) optionally a second further marker gene which is different from the marker gene of iii), second further marker gene being situated within said second expression cassette of ii) or within a further expression cassette, said second further marker gene allowing for the selection of cells comprising said first nucleic acid vector during the process of generating said second nucleic acid vector.

In the methods and kits of the invention, the origins of replication of the first and of the second nucleic acid vectors can be either identical or different.

In the methods and kits of the invention, the target sequence for a recombinase can be the LoxP sequence and the recombinase is then the Cre recombinase.

In the methods and kits of the invention, the markers can be selected from the group consisting of luciferase, β-GAL, CAT, fluorescent protein encoding genes, such as GFP, BFP, YFP, CFP and variants thereof, the lacZα gene, and antibiotics resistance genes, such as chloramphenicol resistance, ampicillin resistance, kanamycin resistance, tetracycline resistance, gentamycin resistance.

In some of the embodiments of the methods and kits of the invention, the first and the second nucleic acid vectors are baculoviruses.

In some embodiments of the invention, the second nucleic acid vector comprises one or more further promoter situated upstream of a target sequence for a recombinase.

In some embodiments of the method of the invention, said method comprises the further steps of transforming cells with the product of the recombination reaction of step c), and selecting for cells expressing the marker gene of iii). The present invention also provides for these cells, said cells being characterized in that they express the marker gene iii), said marker harboring a target sequence for a recombinase as defined in iii) after the promoter driving the expression of the marker gene. In some embodiments, the expression cassette or vector is stably integrated into the genome of said cell.

### BRIEF DESCRIPTION OF THE DRAWINGS

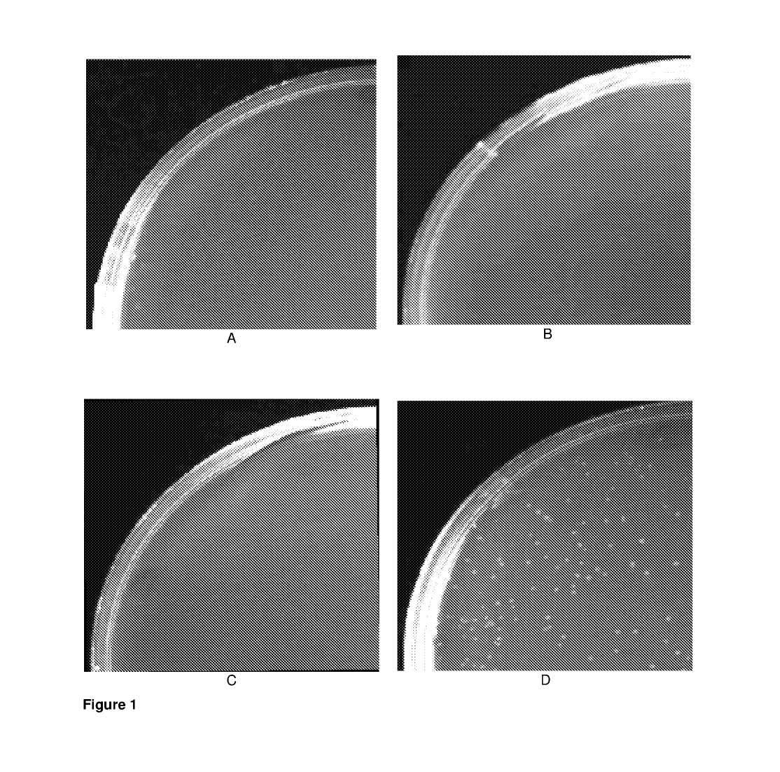

FIG. 1: Different plasmid mixtures were used to transform DH5 alpha E. coli strain using the heat shocking method. Transformed bacteria were plated on LB-agarose medium supplemented with kanamycin at 50 μg/ml final concentration. Plates were observed after incubation at 37° C. during 18 hours
A. Bacteria were transformed with a first nucleic acid plasmid as described in the examples treated with cre recombinase.
B. Bacteria were transformed with a second nucleic acid plasmid as described in the examples treated with cre recombinase.
C. Bacteria were transformed with a mixture of equal amount of the first and the second nucleic acid plasmid. Mixture was not treated by cre recombinase.
D. Bacteria were transformed with a mixture of equal amount of the first and the second nucleic acid plasmid. Mixture was treated by cre recombinase.

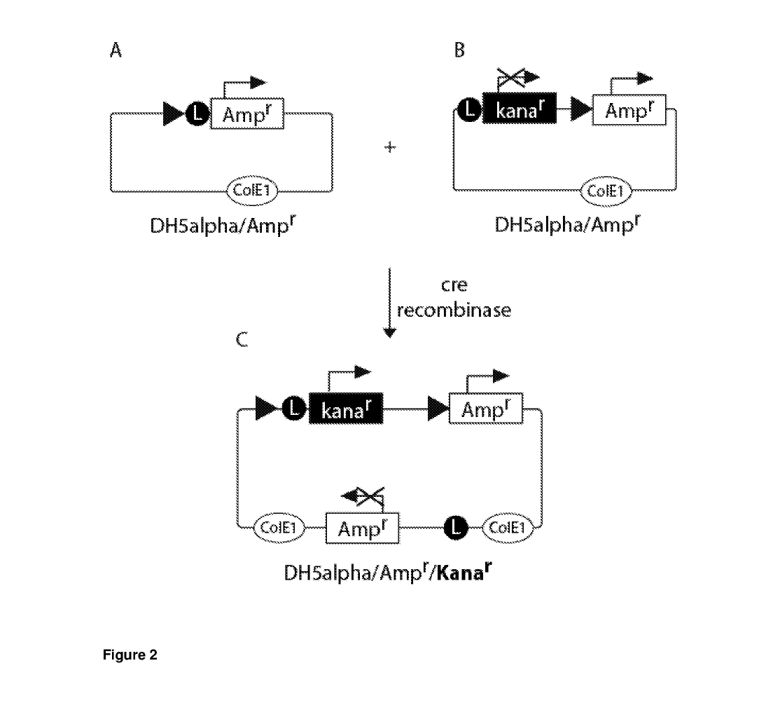

FIG. 2: Schematic representation of the plasmids used in the examples
A. The first plasmid carries the ampicillin promoter sequence shown as a triangle followed by the loxp sequence indicated by the surrounded L letter
B. The second plasmid carries a loxp sequence followed by inactive kana^{r} gene.
C. The plasmid carrying an active kana^{r} gene resulting from cre-loxp fusion of the first plasmid and the second plasmid.

### DETAILED DESCRIPTION OF THE INVENTION

The present inventors have now surprisingly found that it is possible to insert a target sequence for a recombinase between a gene and its promoter, thus allowing the generation of multicistronic nucleic acid vectors.

The present invention hence provides a method for generating polycystronic nucleic acid vectors, said method comprising the steps of a) providing a first nucleic acid vector, said first nucleic acid vector comprising: i) an origin of replication placed in front of ii) at least one first gene of interest functionally cloned within a first expression cassette, said first expression cassette comprising a promoter sequence as well as a termination sequence, said first gene of interest being located between said promoter sequence and said termination sequence, iii) a marker gene together with its termination sequence, said marker gene being situated downstream of a target sequence for a recombinase, wherein the promoter necessary for the expression of said marker gene in a host cell is absent from said first nucleic acid vector, and optionally iv) a first further marker gene which is different from the marker gene of iii), said first further marker gene being situated within said first expression cassette of ii) or within a further expression cassette, said first further marker gene allowing for the selection of cells comprising said first nucleic acid vector during the process of generating said first nucleic acid vector; b) providing a second nucleic acid vector, said second nucleic acid vector comprising i) an origin of replication placed in front of ii) at least one second gene of interest functionally cloned within a second expression cassette, said second expression cassette comprising a promoter sequence as well as a termination sequence, said second gene of interest being located between said promoter sequence and said termination sequence, iii) a promoter situated upstream of the same target sequence for a recombinase as present in front of the marker gene of iii) in the first nucleic acid vector, wherein said promoter is suitable for the expression in a host cell of said marker comprised in the first nucleic acid vector gene, and iv) optionally a second further marker gene which is different from the marker gene of iii), second further marker gene being situated within said second expression cassette of ii) or within a further expression cassette, said second further marker gene allowing for the selection of cells comprising said first nucleic acid vector during the process of generating said second nucleic acid vector; and c) contacting, under conditions suitable for a recombination to take place, a mixture of the first and the second nucleic acid vectors with a recombinase, which recombinase specifically recombines the target sequences of iii) which is situated downstream of the promoter of the second nucleic acid vector and upstream of the marker gene of the first nucleic acid vector, respectively.

The present invention also provides a kit of parts comprising a) a first nucleic acid vector, said first nucleic acid vector comprising i) an origin of replication placed in front of ii) at least one first gene of interest functionally cloned within a first expression cassette, said first expression cassette comprising a promoter sequence as well as a termination sequence, said first gene of interest being located between said promoter sequence and said termination sequence, iii) a marker gene together with its termination sequence, said marker gene being situated downstream of a target sequence for a recombinase, wherein the promoter necessary for the expression of said marker gene in a host cell is absent from said first nucleic acid vector, and iv) optionally a first further marker gene which is different from the marker gene of iii), said first further marker gene situated within said first further marker gene allowing for the selection of cells comprising said first nucleic acid vector during the process of generating said first nucleic acid vector; and b) a second nucleic acid vector, said second nucleic acid vector comprising i) an origin of replication placed in front of ii) at least one second gene of interest functionally cloned within a second expression cassette, said second expression cassette comprising a promoter sequence as well as a termination sequence, said second gene of interest being located between said promoter sequence and said termination sequence, iii) a promoter situated upstream of the same target sequence for a recombinase as present in front of the marker gene in the first nucleic acid vector, wherein said promoter is suitable for the expression in a host cell of the marker comprised in the first nucleic acid vector gene, and iv) optionally a second further marker gene which is different from the marker gene of iii), second further marker gene being situated within said second expression cassette of ii) or within a further expression cassette, said second further marker gene allowing for the selection of cells comprising said first nucleic acid vector during the process of generating said second nucleic acid vector.

In the methods and kits of the invention, the origins of replication of the first and of the second nucleic acid vectors can be either identical or different.

In the methods and kits of the invention, the target sequence for a recombinase can be the LoxP sequence and the recombinase is then the Cre recombinase.

In the methods and kits of the invention, the markers can be selected from the group consisting of luciferase, β-GAL, CAT, fluorescent protein encoding genes, such as GFP, BFP, YFP, CFP and variants thereof, the lacZα gene, and antibiotics resistance genes, such as chloramphenicol resistance, ampicillin resistance, kanamycin resistance, tetracycline resistance, gentamycin resistance.

In some of the embodiments of the methods and kits of the invention, the first and the second nucleic acid vectors are baculoviruses.

In some embodiments of the invention, the second nucleic acid vector comprises one or more further promoter situated upstream of a target sequence for a recombinase.

In some embodiments of the method of the invention, said method comprises the further steps of transforming cells with the product of the recombination reaction of step c), and selecting for cells expressing the marker gene of iii). The present invention also provides for these cells, said cells being characterized in that they express the marker gene iii), said marker harboring a target sequence for a recombinase as defined in iii) after the promoter driving the expression of the marker gene. In some embodiments, the expression cassette or vector is stably integrated into the genome of said cell.

As used herein, a “vector” is a molecule used as a vehicle to transfer foreign genetic material into a cell. The four major types of vectors are plasmids, viral vectors, cosmids, and artificial chromosomes. Vectors comprise at least an origin of replication, a multicloning site, and a selectable marker. The vector itself is generally a DNA sequence that consists of an insert (transgene) and a larger sequence that serves as the “backbone” of the vector. The purpose of a vector which transfers genetic information to another cell is typically to isolate, multiply, or express the insert(s) in the target cell. Vectors called “expression vectors” (“expression constructs”) are for the expression of the transgene in the target cell, and generally have a promoter sequence that drives expression of the transgene. Simpler vectors called transcription vectors are only capable of being transcribed but not translated: they can be replicated in a target cell but not expressed, unlike expression vectors. Transcription vectors are used to amplify their insert.

Insertion of a vector into the target cell is usually called “transformation” for bacterial cells, “transfection” for eukaryotic cells, although insertion of a viral vector is often called “transduction”. As used herein, transformation, transfection and transduction are interchangeable terms. “Viral vectors” are genetically-engineered viruses carrying modified viral DNA or RNA that has been rendered noninfectious, but still contain viral promoters and also the transgene, thus allowing for translation of the transgene through a viral promoter. It is well known to the skilled person that because viral vectors frequently are lacking infectious sequences, they require helper viruses or packaging lines for large-scale transfection. Viral vectors are often designed for permanent incorporation of the insert into the host genome, and thus leave distinct genetic markers in the host genome after incorporating the transgene. For example, retroviruses leave a characteristic retroviral integration pattern after insertion that is detectable and indicates that the viral vector has incorporated into the host genome. Examples of viral vectors are retroviruses, lentiviruses, adenoviruses, adeno-associated viruses (AAV) and baculoviruses. “Baculoviruses” are a family of large rod-shaped viruses that can be divided to two genera: nucleopolyhedroviruses (NPV) and granuloviruses (GV). While GVs contain only one nucleocapsid per envelope, NPVs contain either single (SNPV) or multiple (MNPV) nucleocapsids per envelope. The enveloped virions are further occluded in granulin matrix in GVs and polyhedrin for NPVs. Moreover, GV have only single virion per granulin occlusion body while polyhedra can contain multiple embedded virions. Baculoviruses have very species-specific tropisms among the invertebrates with over 600 host species having been described. Immature (larval) forms of moth species are the most common hosts, but these viruses have also been found infecting sawflies, mosquitoes, and shrimp. Although baculoviruses are capable of entering mammalian cells in culture, they are not known to be capable of replication in mammalian or other vertebrate animal cells. Baculoviruses contain circular double-stranded genome ranging from 80-180 kbp. Baculovirus expression in insect cells represents a robust method for producing recombinant glycoproteins. Baculovirus-produced proteins are currently under study as therapeutic cancer vaccines with several immunologic advantages over proteins derived from mammalian sources. The baculovirus expression system has been used extensively for the expression of recombinant proteins in insect cells. The viruses can be readily manipulated, accommodate large insertions of foreign DNA, initiate little to no microscopically observable cytopathic effect in mammalian cells and have a good biosafety profile (Kost et al., 2002, Trends in Biotechnology, 20(4)). These attributes make baculoviruses particularly useful for practicing the present invention.

As used herein, the term “promoter” refers to any cis-regulatory elements, including enhancers, silencers, insulators and promoters.

As used herein, the term “expression cassette” is to be understood to relate to a DNA fragment which contains a promoter sequence used to recruit the DNA transcription machinery, followed by an oligonucleotide encoding for a signal sequence to recruit the RNA translation machinery (e.g. Kozak consensus in eukaryotes or Shide-Dalgamo in prokaryotes), followed by an oligonucleotide sequence which contains at least one DNA sequence cleaved by a restriction enzyme (multiple cloning site MCS) to be used for insertion of DNA fragments encoding for gene products of choice, also termed “gene of interest”, and a terminator sequence which directs the processes related to the end of transcription and modification of the RNA transcript (e.g. polyadenylation in eukaryotes).

As used herein, the term “multiplication module” is to be understood to relate to a set of DNA fragments placed outside of expression cassettes, and/or in between expression cassettes, that allow for iterative combination of several or many expression cassettes.

Compatible restriction sites are restriction sites that, when cleaved by the cognate enzymes, result in overhanging or recessing single-stranded regions of DNA of the same nucleotide length which are cohesive, i.e. they can anneal to each other using complementary Watson-Crick base pairing, and thus can be rejoined by ligases to yield a covalently linked functional DNA molecule. Alternatively, restriction sites that when cleaved result in blunt-ended DNA fragments, are also compatible, i.e. they can be rejoined by ligases to yield covalently linked functional DNA molecules. Non-limiting examples of restriction endonucleases that produce compatible overhangs are SpeI/AvrII; AgeI/XmaI/SgrA; BamHI/BglII; BsrGI/BanI; EagI/NotI; EcoRI/MfeI; NdeI/AseI; NheI/XbaI; PstI/NsiI; SalI/XhoI. Non-limiting examples of restriction endonucleases that produce compatible blunt ends, e.g. all combinations can be ligated to each other, are BstZ17I, EcoRV, FspI, HpaI, MluNI, PmeI, SeaI, SnaBI, StuI, XmnI.

The terms “head to head”, “head to tail”, and “tail to tail” define the arrangement of the promoters and terminators of the individual expression cassettes towards each other. For example, terminator-terminator is a “tail to tail” arrangement, promoter-promoter is a “head to head” arrangement, and promoter-terminator is a “head to tail” arrangement.

Expression cassettes are typically introduced into a vector that facilitates entry of the expression cassette into a host cell and maintenance of the expression cassette in the host cell. Such vectors are commonly used and are well known to those of skill in the art. Numerous such vectors are commercially available, e. g., from Invitrogen, Stratagene, Clontech, etc., and are described in numerous guides, such as Ausubel, Guthrie, Strathem, or Berger, all supra. Such vectors typically include promoters, polyadenylation signals, etc. in conjunction with multiple cloning sites, as well as additional elements such as origins of replication, selectable marker genes (e. g., LEU2, URA3, TRP 1, HIS3, GFP), centromeric sequences, etc.

As used herein, the term “animal” is used herein to include all animals. In some embodiments of the invention, the animal is a non-human vertebrate. Examples of animals are human, mice, rats, cows, pigs, horses, chickens, ducks, geese, cats, dogs, fruit flies, fishes, etc. The term “animal” also includes an individual animal in all stages of development, including embryonic and fetal stages. A “genetically-modified animal” is any non-human animal containing one or more cells bearing genetic information altered or received, directly or indirectly, by deliberate genetic manipulation at a sub-cellular level, such as by targeted recombination, microinjection or infection with recombinant virus. The term “genetically-modified animal” is not intended to encompass classical crossbreeding or in vitro fertilization, but rather is meant to encompass non-human animals in which one or more cells are altered by, or receive, a recombinant DNA molecule. This recombinant DNA molecule may be specifically targeted to a defined genetic locus, may be randomly integrated within a chromosome, or it may be extrachromosomally replicating DNA. The term “germ-line genetically-modified animal” refers to a genetically-modified non-human animal in which the genetic alteration or genetic information was introduced into germline cells, thereby conferring the ability to transfer the genetic information to its offspring. If such offspring in fact possess some or all of that alteration or genetic information, they are genetically-modified animals as well.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically. “Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons). In some embodiments, the “gene” does not comprise introns.

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=I, Window Size=sequence length, Gap Penalty—5, Gap Size Penalty—0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

“Fluorescence” refers to any detectable characteristic of a fluorescent signal, including intensity, spectrum, wavelength, intracellular distribution, etc.

“Detecting” fluorescence refers to assessing the fluorescence of a cell using qualitative or quantitative methods. In some of the embodiments of the present invention, fluorescence will be detected in a qualitative manner. In other words, either the fluorescent marker is present, indicating that the recombinant fusion protein is expressed, or not. For other instances, the fluorescence can be determined using quantitative means, e. g., measuring the fluorescence intensity, spectrum, or intracellular distribution, allowing the statistical comparison of values obtained under different conditions. The level can also be determined using qualitative methods, such as the visual analysis and comparison by a human of multiple samples, e. g., samples detected using a fluorescent microscope or other optical detector (e. g., image analysis system, etc.). An “alteration” or “modulation” in fluorescence refers to any detectable difference in the intensity, intracellular distribution, spectrum, wavelength, or other aspect of fluorescence under a particular condition as compared to another condition. For example, an “alteration” or “modulation” is detected quantitatively, and the difference is a statistically significant difference. Any “alterations” or “modulations” in fluorescence can be detected using standard instrumentation, such as a fluorescent microscope, CCD, or any other fluorescent detector, and can be detected using an automated system, such as the integrated systems, or can reflect a subjective detection of an alteration by a human observer.

The “green fluorescent protein” (GFP) is a protein, composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostablility and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ε), also known as its optical cross section of 9.13×10-21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm.

As used herein, the singular forms “a”, “an,” and the include plural reference unless the context clearly dictates otherwise.

A “virus” is a sub-microscopic infectious agent that is unable to grow or reproduce outside a host cell. Each viral particle, or virion, consists of genetic material, DNA or RNA, within a protective protein coat called a capsid. The capsid shape varies from simple helical and icosahedral (polyhedral or near-spherical) forms, to more complex structures with tails or an envelope. Viruses infect cellular life forms and are grouped into animal, plant and bacterial types, according to the type of host infected.

Preferred SSRs (site specific recombinases) are the cre-lox specific recombination (LoxP) site or the FLP recombinase specific recombination (FRT) site.

A “marker” or “marker gene” is a gene whose expression product allows to differentiate between cells expressing it and cells which do not express it. Such a marker can be selected from the group consisting of luciferase, β-GAL, CAT, fluorescent protein encoding genes, such as GFP, BFP, YFP, CFP and variants thereof, the lacZα gene, antibiotics resistance genes, such as chloramphenicol resistance, ampicillin resistance, kanamycin resistance, tetracycline resistance, gentamycin resistance. Variants of marker genes are those that deviate in sequence by less than 30, preferably less than 20, more preferably less than 10% but retain substantially the same functional marker properties as the original marker gene.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Examples

As a first plasmid, a plasmid of 6.7 kbp carrying a bacterial transcription promoting sequence, the so called ampicillin promoter (TTCAAATATGTATCCGCTCATGAGACAAT SEQ ID NO:1), followed by loxp sequence was used.

As a second plasmid, a plasmid of 7.3 kbp carrying a loxp sequence followed by the gene of bacterial Kanamycin resistance was used. In the absence of a bacterial transcription promoting sequence placed in 5′, the Kanamycin resistance gene (kana^{r}) is inactive.

In this example, both plasmids carry an active (placed under control of a bacterial promoter) ampicillin resistance gene and carry the ColE1 replication origin. Both plasmids can be handled and/or amplified in DH5alpha E. coli strain grown in the same conditions in presence of ampicillin at 100 μg/ml final concentration. One hundred nanograms of each plasmid were mixed and treated with 1 unit of purified recombinant cre recombinase (New England Biolabs®; reference: M0298S) in 10 μl final volume for 25 minutes at 37° C. in cre recombinase buffer 1× (New England Biolabs®). This reaction mixture has then been incubated at 70° C. for 15 minutes to inactivate the enzyme. The entire reaction mixture has then been used to transform RbCl₂ chimio-competent cells by heat shock as known in the art. The transformed cells have then been mixed with SOC medium and incubated at 37° C. under shaking conditions for 2 hours prior to plating on LB-agarose plates supplemented with kanamycin at 50 μg/ml final concentration. The plates were incubated at 37° C. for 24 hours. Colonies Controls included the treatment of only the first plasmid treated with cre recombinase, of only the second plasmid treated with cre recombinase, and the mixture of both plasmids without cre recombinase treatment.

1. A method for generating polycystronic nucleic acid vectors, said method comprising the steps of:
a) providing a first nucleic acid vector, said first nucleic acid vector comprising:
i) an origin of replication placed in front of
ii) at least one first gene of interest functionally cloned within a first expression cassette, said first expression cassette comprising a promoter sequence as well as a termination sequence, said first gene of interest being located between said promoter sequence and said termination sequence,
iii) a marker gene together with its termination sequence, said marker gene being situated downstream of a target sequence for a recombinase, wherein the promoter necessary for the expression of said marker gene in a host cell is absent from said first nucleic acid vector, and
iv) optionally a first further marker gene which is different from the marker gene of iii), said first further marker gene being situated within said first expression cassette of ii) or within a further expression cassette, said first further marker gene allowing for the selection of cells comprising said first nucleic acid vector during the process of generating said first nucleic acid vector;
b) providing a second nucleic acid vector, said second nucleic acid vector comprising:
i) an origin of replication placed in front of
ii) at least one second gene of interest functionally cloned within a second expression cassette, said second expression cassette comprising a promoter sequence as well as a termination sequence, said second gene of interest being located between said promoter sequence and said termination sequence,
iii) a promoter situated upstream of the same target sequence for a recombinase as present in front of the marker gene of iii) in the first nucleic acid vector, wherein said promoter is suitable for the expression in a host cell of said marker comprised in the first nucleic acid vector gene, and
iv) optionally a second further marker gene which is different from the marker gene of iii), second further marker gene being situated within said second expression cassette of ii) or within a further expression cassette, said second further marker gene allowing for the selection of cells comprising said first nucleic acid vector during the process of generating said second nucleic acid vector;
c) contacting, under conditions suitable for a recombination to take place, a mixture of the first and the second nucleic acid vectors with a recombinase, which recombinase specifically recombines the target sequences of iii) which is situated downstream of the promoter of the second nucleic acid vector and upstream of the marker gene of the first nucleic acid vector, respectively.

2. Kit of parts comprising:
a) a first nucleic acid vector, said first nucleic acid vector comprising:
i) an origin of replication placed in front of
ii) at least one first gene of interest functionally cloned within a first expression cassette, said first expression cassette comprising a promoter sequence as well as a termination sequence, said first gene of interest being located between said promoter sequence and said termination sequence,
iii) a marker gene together with its termination sequence, said marker gene being situated downstream of a target sequence for a recombinase, wherein the promoter necessary for the expression of said marker gene in a host cell is absent from said first nucleic acid vector, and
iv) optionally a first further marker gene which is different from the marker gene of iii), said first further marker gene situated within said first further marker gene allowing for the selection of cells comprising said first nucleic acid vector during the process of generating said first nucleic acid vector;
and
b) a second nucleic acid vector, said second nucleic acid vector comprising:
i) an origin of replication placed in front of
ii) at least one second gene of interest functionally cloned within a second expression cassette, said second expression cassette comprising a promoter sequence as well as a termination sequence, said second gene of interest being located between said promoter sequence and said termination sequence,
iii) a promoter situated upstream of the same target sequence for a recombinase as present in front of the marker gene in the first nucleic acid vector, wherein said promoter is suitable for the expression in a host cell of the marker comprised in the first nucleic acid vector gene, and
iv) optionally a second further marker gene which is different from the marker gene of iii), second further marker gene being situated within said second expression cassette of ii) or within a further expression cassette, said second further marker gene allowing for the selection of cells comprising said first nucleic acid vector during the process of generating said second nucleic acid vector.

3. The method of claim 1, wherein the origins of replication of the first and of the second nucleic acid vectors are identical.

4. The method of claim 1, wherein the target sequence for a recombinase is the LoxP sequence and the recombinase is the Cre recombinase,

5. The kit of claim 2, wherein the target sequence for a recombinase is the LoxP sequence.

6. The method of claim 1, wherein the markers are selected from the group consisting of luciferase, β-GAL, CAT, fluorescent protein encoding genes, such as GFP, BFP, YFP, CFP and variants thereof, the lacZα gene, and antibiotics resistance genes, such as chloramphenicol resistance, ampicillin resistance, kanamycin resistance, tetracycline resistance, gentamycin resistance.

7. The method of claim 1, wherein the first and the second nucleic acid vectors are baculloviruses.

8. The method of claim 1, wherein said second nucleic acid vector comprises one or more further promoter situated upstream of a target sequence for a recombinase.

9. The method of claim 1, further comprising the steps of:
d) transforming cells with the product of the recombination reaction of step c), and
e) selecting for cells expressing the marker gene of iii).

10. An isolated cell obtained by the method of claim 9, said cell being characterized in that it expresses the marker gene defined in iii) of claim 1, said marker harboring a target sequence for a recombinase as defined in iii) of claim 1 after the promoter driving the expression of the marker gene.

11. The cell of claim 9 wherein the expression cassette or vector is stably integrated into the genome of said cell.
